(19) United States
(12) Patent Application Publication
(10) Pub. No.: US 2011/0008822 A1
(43) Pub. Date: Jan. 13, 2011

(54) CASSETTE AND METHOD FOR PLANT SAMPLING

(76) Inventors: Allan David Morrison, Lota Queensland (AU); Bradley F. Davin, Seneca, SC (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 12/919,373

(22) PCT Filed: Feb. 26, 2009
(86) PCT No.: PCT/AU09/00220

§ 371 Date: Aug. 25, 2010

Related U.S. Application Data
(60) Provisional application No. 61/031,498, filed on Feb. 26, 2008.

(51) Int. Cl. C12Q 1/02; C12M 3/00

(52) U.S. Cl. 435/29; 435/307.1

(57) ABSTRACT

A method and storage medium (100) for collecting, storing and analysing a plant sample. The storage medium (100) is configured to provide for multiple portions of the sample to be withdrawn from the storage medium (100) by moving a punch through one of a plurality of apertures (120) through the lid (102) and base (104) of the storage medium. The storage medium includes a gripping region on its interior surface (110) to grip a sample between the lid (102) and the base (104) of the storage medium (100).

### FIELD OF THE INVENTION

The present invention relates to improvements in devices designed to capture and store plant samples for later analysis.

### BACKGROUND OF THE INVENTION

A problem often encountered in the collection of plant samples is the capture of a plant sample in a way that lends itself to easy storage and analysis. Existing methods of capturing and storing plant samples are labour intensive and inadequate for maximising the use of the sample for later analysis. Accordingly there exists a need which reduces one or more problems of known plant collection systems and methods.

### SUMMARY

The present invention in one preferred aspect provides for a device and method adapted to capture and store a sample in a way that lends itself to a greater utilisation of the sample, and preferably permits improved automation of plant sample analysis.

### BRIEF DESCRIPTION OF THE FIGURES

FIG. 1 is a perspective view of a plant sample storage cassette in an open position in accordance with a preferred embodiment of the present invention.

FIG. 1A is a partial enlarged perspective view of a hinge connecting the lid and base of the plant sample storage cassette taken along line A.

FIG. 1B is a partial enlarged perspective view of a portion of the front of the base of the plant sample storage cassette taken along line B.

FIG. 2 is a perspective view of the plant sample storage cassette of FIG. 1 in a closed position.

FIG. 2A is a partial enlarged perspective view of the front of the lid and base of the plant sample storage cassette taken along line A.

FIG. 3 is a perspective view of a plant sample storage cassette in an open position in accordance with another preferred embodiment of the present invention.

FIG. 4 is a perspective view of the plant sample storage cassette of FIG. 3 in a closed position.

FIG. 5 is a perspective view of a plant sample storage cassette in a closed position in accordance with another preferred embodiment of the present invention.

FIG. 6 is a perspective view of a plant sample storage cassette in an open position in accordance with another preferred embodiment of the present invention.

FIG. 7 is a partial expanded side cross sectional view of the interior of the plant sample storage cassette of FIG. 6 in a closed position.

FIG. 8 is a partial expanded side, cross sectional view of a gripping/cutting surface of the interior of the plant sample storage cassette in a closed position in accordance with another preferred embodiment of the present invention.

FIG. 9 is a top view of an aperture configuration in accordance with another preferred embodiment of the present invention.

### DETAILED DESCRIPTION OF THE DRAWINGS

Alternative embodiments of the invention will be apparent to those skilled in the art from consideration of the specification and practice of the invention disclosed herein. It is intended that the specification and examples be considered as exemplary only, with a true scope and spirit of the invention being indicated by the claims which follow.

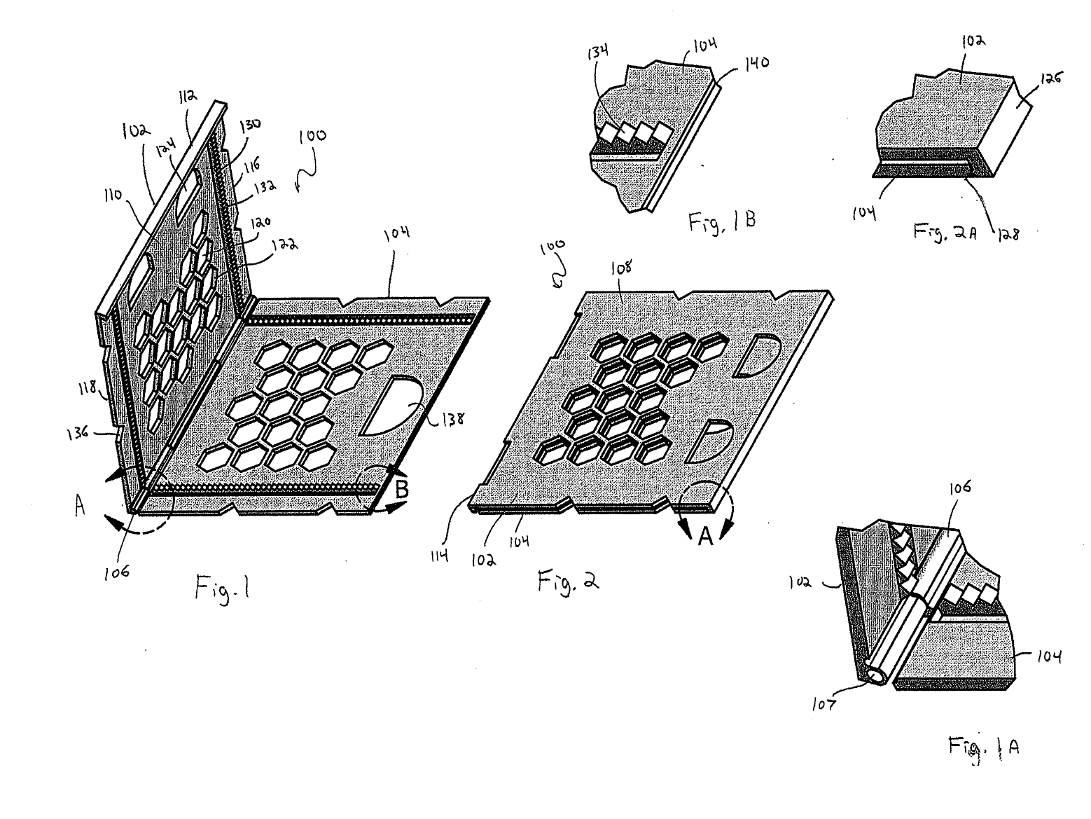

FIGS. 1 to 2A show a preferred embodiment of a plant sample storage medium preferably in the form of a cassette 100. Cassette 100 preferably includes a lid 102 and a base 104 connected for moveable engagement by a hinge 106. The preferred elements of cassette 100 and their interrelationship are described below.

As shown in FIG. 1A, lid 102 and base 104 are preferably moveably connected to one another by hinge 106. Hinge 106 may be formed in a variety of ways. For example, as shown in FIG. 1A, hinge 106 includes a channel 107 formed by portions of lid 102 and base 104. Channel 107 is sized to receive a pin. Hinge 106 may be configured as an integral portion of lid 102 and base 104 if desired, such as a living hinge.

Referring to FIGS. 1 and 2, lid 102 includes an exterior surface 108, an interior surface 110, a front 112, a rear 114 and sides 116, 118. Lid 102 preferably includes a plurality of apertures 120 through exterior surface 108 and interior surface 110. Apertures 120 are preferably hexagonally shaped and sized to receive a punch therethrough. It will be appreciated that apertures 120 may be shaped in a variety of ways without departing from the scope of the present invention. The number, placement and shape of the apertures may be varied without departing from the scope of the invention. For example, apertures 120 may be polygonal (square, rectangular, etc), circular, oval or a combination thereof.

In a preferred form, apertures 120 form a lattice structure 122 that occupies a substantial portion, preferably a majority of the lid. The structure forming lattice 120 is preferably sufficiently strong enough to withstand the interaction of a punch with a plant sample stored within cassette 100.

Lid 102 preferably includes a pair of generally semi-circular finger openings 124 proximate front 112 to assist a user in opening and closing the cassette. It will be appreciated that the number, placement and shape of openings 124 may be varied, or omitted as desired. As shown in FIG. 2A, lid 102 preferably includes a resilient tab 126 having a surface deviation 128 along the length thereof. Tab 126 is configured for snap-fit engagement with a corresponding surface deviation in the base, further described below.

As shown in FIG. 1, interior surface 110 preferably includes a gripping region which in a preferred form includes two rows 130 of surface deviations 132. FIG. 1B shows surface deviations 132 that are preferably shaped as ratchetings 134 having front and back faces that are preferably at a 90 degree angle relative to one another. The number, placement, and shape of the gripping region may be varied without departing from the scope of the invention. For example, surface projections may be dispersed throughout the interior surface instead of arrayed along parallel rows. The surface deviations may be arrayed along one, two, three or four sides of the interior surface. The surface deviations may be shaped as conical, frusto-conical, domed, pyramidal or pin-shaped. When ratchetings are used, the forward and rearward faces may be at steeper or shallower angles relative to one another. The ratchetings may all face the same direction, or groups of ratchetings may be formed facing different directions. The surface deviations are preferably configured to interact with a corresponding set of surface deviations on base 104 to grip a plant sample between lid 102 and base 104. A chemical agent adapted to function as an adhesive may replace or supplement the surface deviations.

Sides 116, 118 each preferably include a pair of indentations 136 which assist in the handling of cassette 100 with automated instrumentation and storage facilities. Indentations 136 may be configured in a variety of ways or omitted if desired.

In a preferred form the structure of base 104 is similar to that of lid 102, so unless otherwise noted, the description of lid 102 will be understood to apply to base 104 as appropriate. Base 104 is essentially the same as lid 102 except that instead of finger openings 124, base 104 includes a semi-circular finger opening 138 proximate the front of base 104. Finger opening 138 is preferably configured to assist a user in the opening and closing of cassette 100 in conjunction with finger openings 124. Like openings 124, it will be appreciated that the number, placement and shape of opening 138 may be varied, or omitted as desired.

As shown in FIGS. 1B and 2A, base 104 preferably includes an indent or groove 140 along the length of the front to engage with surface deviation 128 of tab 126 of lid 102 for snap-fit engagement between lid 102 and base 104. Lid 102 and base 104 may be secured together when closed in a variety of ways other than snap-fit engagement. For example only, the lid and base may include magnets, Velcro and a variety of interacting resilient tab or projection configurations.

Having described the preferred components of cassette 100, a preferred method of use will now be described with reference to FIGS. 1 and 2. A user inserts a portion of a plant between the interior surfaces of lid 102 and base 104 to obtain a sample of the plant with the cassette in an open position (FIG. 1). The lid and base are moved to a closed position (FIG. 2) by pivoting the lid and base about hinge 106 to trap the plant sample in cassette 100. Tab 126 is engaged with groove 140 to secure or lock the lid and base into the closed position. The cassette is inserted into a carrier or magazine for transfer to a location where the sample may be analysed. Once at the location (such as a lab), the cassette is withdrawn from the carrier. A punching device is used to punch or otherwise remove a portion of the sample from the cassette. The punch is preferably inserted through the cassette along the central longitudinal axis of a punching channel formed by the alignment of apertures 120 of lid 102 and base 104. The punched disc portion of the sample is collected and analysed as needed. The cassette is returned to storage for any later analysis as needed. The cassette may be further withdrawn from storage and additional portions punched or otherwise removed from the cassette as needed. Preferably, the sample is kept in a cool environment to inhibit degradation. To this end, the cassette or carrier may be stored in a container adapted to maintain the sample in a cooler environment.

The sample may be identified by applying an identifier to the cassette. Examples of identifiers include, but are not limited to barcodes, symbols or other indicia. The identifier facilitates automated analysis of the sample by, for example, permitting a device to retrieve a cassette from a magazine and transfer the cassette to a location where the sample may be punched or otherwise removed from the cassette.

It will be appreciated that the steps described above may be performed in a different order, varied, or omitted entirely without departing from the scope of the present invention. Examples of punches or sample analysis systems are described in one or more of U.S. application Ser. No. 11/676,253, entitled “Biological Sample Collection Device;” U.S. application Ser. No. 10/982,539, entitled “System and Method for Analysing Laboratory Samples;” U.S. application Ser. No. 11/148,094, entitled “Method and Apparatus for Inspecting Biological Samples;” International Application No. PCT/AU2007/000171, entitled “Biological Sample Collection Device;” and International Application No. PCT/AU99/00485, entitled “a Punching Apparatus,” the disclosure of each being incorporated herein by reference.

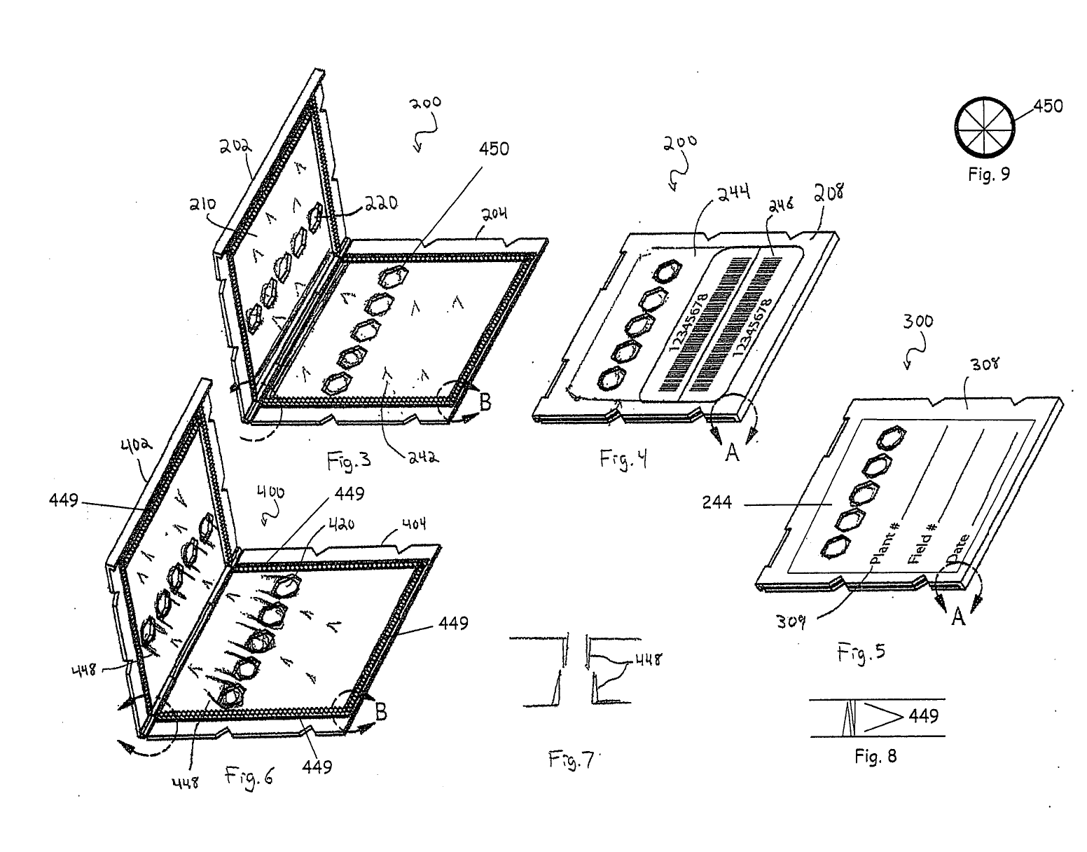

FIGS. 3 and 4 show another preferred embodiment of a plant sample storage medium preferably in the form of a cassette 200. Cassette 200 is similar to cassette 100 except that cassette 200 preferably includes an additional and more centralised gripping region on the interior surface 210, and apertures 220 are configured along a single row instead of a lattice. In a preferred form, the central gripping region includes a plurality of holding pins 242. Pins 242 may vary in number, placement and shape as desired. For example, pins 242 may be configured as conical, pyramidal, or any other suitable shape. Preferably, pins 242 are arranged on lid 202 and base 204 so as to be directly opposed to each other to trap or hold a sample therebetween. Pins 242 on lid 202 and base 204 may be offset from one another relative to the vertical plane if desired.

As shown in FIG. 4, a label 244 may be added to exterior surface 208 if desired. Label 244 is preferably sized and shaped to cover a substantial portion of the exterior surface of lid 202, including apertures 220. Label 244 is preferably manufactured from a breathable material to allow air communication through apertures 220. Label 244 further preferably includes one or more identifiers such as bar codes 246. It will be appreciated that the label is optional and may be configured in a variety of ways, if included with the cassette. For example only, exterior surface 208 may be indented so that a label may be applied to a portion of the indentation. The label may be formed as part of a sleeve configured to slide over cassette 200 if desired. The label may be breathable or impermeable depending upon the requirements needed for the cassette. The label may include one or more flaps or fingers to interact with a portion of the cassette. The label may be applied to the interior surface, the exterior surface, or both the interior and exterior surface of the cassette if desired.

FIG. 5 shows another preferred embodiment of a plant sample storage medium preferably in the form of a cassette 300. Cassette 300 is similar to cassette 200 except that cassette 300 preferably includes an identifier 309 that is printed on exterior surface 308. Similar to cassette 200, identifier 309 may be printed on the interior surface, the exterior surface, or both the interior and exterior surface of the cassette if desired. Identifier 309 may contain information specific to the sample collected and may be configured for filling in by a person, have bar code information, or both. Additionally, a sleeve such as described above may be used with cassette 300 if needed.

FIGS. 6 and 7 show another preferred embodiment of a plant sample storage medium preferably in the form of a cassette 400. Cassette 400 is similar to cassette 200 except that cassette 400 preferably includes a cutting projection 448 centered around the central longitudinal axis of each aperture 420. As shown in FIG. 7, cutting projections 448 are preferably slightly offset in the horizontal plane so that the cutting projections located on the interior surface of lid 402 and base 404 will cut a portion of any sample in cassette 400, similar to opposed, differently sized cookie cutters moving towards one another. Cutting projections 448 are preferably cylindrical and have an approximate circumference of 1.5 mm. It will be appreciated that the cutting projections may be formed with other shapes/cross sections, such as, but not limited to oval, elliptical, square, etc. The foregoing description is by way of example only, and may be varied considerably without departing from the scope of the present invention. For example only, as shown in FIG. 2, cassette 100 is generally a flat, square-shaped container. It will be appreciated that the storage medium may be formed in a variety of ways, for example, as a rectangular wafer or cylinder. The interior of the storage medium preferably includes a gripping region which may be structural, chemical or a combination thereof.

The gripping region may include a cutting surface in lieu of or in addition to surface deviations 132. For example, the opposed interior surfaces of lid 102 and base 104 may include a cutting surface 449 (FIG. 8) so that when lid 102 and base 104 are moved to a closed position, the cutting surface functions similar to the cutting action of scissors to cut a sample to substantially the shape of the cassette. The gripping and/or cutting surface may extend around two, three or four sides of the cassette. It will be appreciated that the gripping/cutting surface may follow the perimeter of the cassette and may be curved or interrupted as desired.

As shown in FIG. 9, the apertures in the lid and/or base may include a plurality of pie-shaped flaps 450 integrally formed with the cassette. If desired, the sleeve mentioned above with respect to cassette 200 may be formed with perforated flaps configured to generally coincide with the apertures of a cassette over which the sleeve is adapted to fit over. Such a configuration beneficially permits a user to locate the apertures and access the apertures as needed if the sleeve material is of a nature where it would be difficult to discern the location of the apertures while the sleeve covers the cassette.

It will be appreciated that the storage medium may be made from a variety of materials such as plastic, metal, or a combination thereof without departing from the scope of the present invention. The storage medium is preferably constructed of a material adapted to withstand the handling that may be expected in transferring the cassette and obtaining portions of samples therefrom.

The features described with respect to one embodiment may be applied to other embodiments, or combined with or interchanged with the features other embodiments, as appropriate, without departing from the scope of the present invention.

The present invention in a preferred form provides one or more of the advantages set forth below. For example, the configuration of the storage medium provides more efficient handling and use by automated systems. The configuration of the storage medium also has improved storage which allows for multiple portions of the sample to be taken from the same storage medium. The shape of the storage medium permits more samples to be stored in a smaller space. The combination of the storage medium with an identifier permits a user or automated system to identify and retrieve a sample with relative ease. The lattice structure permits the sample within the cassette to be cooled more quickly and more evenly, lessening the rate of degradation of the sample. The storage medium is preferably configured to permit multiple portions of the sample within the medium to be removed from the storage medium without opening the storage medium.

The system and method of the present invention may be used in a variety of applications. For example, the cassette is well suited for collection, storage and analysis of leaf samples. It will be appreciated that the system and method of the present invention may be conformed to application in other plant samples as needed.

It will of course be realised that the above has been given only by way of illustrative example of the invention and that all such modifications and variations thereto as would be apparent to persons skilled in the art are deemed to fall within the broad scope and ambit of the invention as herein set forth.

The claims defining the invention are as follows:

1. A method for analysing a plant sample, comprising:
inserting at least a portion of a plant between first and second surfaces of a storage medium to obtain a sample of the plant; and
moving a punch through a section of each of the first and second surfaces to remove a portion of the sample from the storage medium.

2. The method of claim 1, further comprising cooling the sample after the portion of the sample has been removed.

3. The method of either claim 1 or 2, further comprising moving the first and second surfaces toward one another to capture the sample therebetween.

4. The method of claim 3, wherein the step of moving includes pivoting the first and second surfaces relative to one another.

5. The method of any one of the preceding claims, wherein the section of each of the first and second surfaces includes an aperture sized to receive the punch.

6. The method of any one of the preceding claims, further comprising moving the punch through another section of each of said first and second surfaces to remove a further portion of the sample from the storage medium.

7. The method of any one of the preceding claims, further comprising the step of assigning an identifier to the storage medium to identify the sample in the storage medium.

8. The method of claim 7, wherein the step of assigning includes attaching a barcode to a portion of the storage medium.

9. A plant sample storage medium, comprising:
a lid having an interior surface and a plurality of apertures through said lid;
a base moveably connected to said lid, said base having an interior surface and a plurality of apertures through said base that generally align with said plurality of apertures through said lid to define a punch channel, said interior surfaces of said lid and said base each including a region configured to grasp hold of a sample inserted between said lid and said base when said lid and said base are moved towards a closed position.

10. The sample storage medium of claim 9, wherein said lid and said base are hingedly connected.

11. The sample storage medium of either claim 9 or 10, wherein said apertures of said lid occupy a majority of the area of said lid.

12. The sample storage medium of any one of claims 9 to 11, wherein said apertures of said lid and said base are non-circular.

13. The sample storage medium of any one of claims 9 to 12, wherein said apertures of said lid and said base are hexagonally shaped.

14. The sample storage medium of any one of claims 9 to 13, wherein said region of said lid and said base includes a plurality of surface deviations.

15. The sample storage medium of claim 14, wherein said surface deviations includes a plurality of ratchets.

16. The sample storage medium of any one of claims 9 to 15, wherein said grasping region of said lid and said base is oriented in a row generally parallel to a side of said lid and said base, respectively.

17. The sample storage medium of claim 16, wherein said grasping region of said lid and said base is generally oriented along two parallel rows.

18. The sample storage medium of any one of claims 9 to 17, wherein said lid and said base are configured to lock together when said sample storage medium is in the closed position.

19. The sample storage medium of 18, wherein a front portion of said lid and said base are configured to snap fit with each other.

20. The sample storage medium of any one of claims 9 to 19, further comprising an identifier to identify the sample stored in said sample storage medium.

21. The sample storage medium of claim 20, wherein said identifier includes a barcode.

22. The sample storage medium of any one of claims 9 to 21, wherein said lid and said base are each generally planar.

23. The sample storage medium of any one of claims 9 to 22, wherein said lid and said base each have a generally square outside perimeter.

24. The sample storage medium of claims 9 to 23, wherein said grasping region includes a plurality of pins.

25. The sample storage medium of claim 24, wherein said pins are directly opposed to one another.

26. The sample storage medium of any one of claims 9 to 13, wherein said grasping region includes a cutting edge generally aligned along a majority of the perimeter of said storage medium.

27. The sample storage medium of any one of claims 9 to 13, wherein each of said lid and said base include a cutting edge configured to cut a portion of a sample therebetween.

28. The sample storage medium of claim 9, wherein at least one of said apertures includes a cutting projection oriented towards the interior of said storage medium.

29. The sample storage medium of any one of claims 9 to 28, further comprising a sleeve.

30. The sample storage medium of claim 29, wherein said sleeve is made of a breathable material.

31. The sample storage medium of either claim 29 or 30, wherein said sleeve includes an identifier.
